(19) United States
(12) Patent Application Publication
(10) Pub. No.: US 2026/0217682 A1
(43) Pub. Date: Jul. 30, 2026

(54) STILBENE COMPOUND AND PREPARATION METHOD THEREFOR AND USE THEREOF

(71) Applicant: THE HONG KONG UNIVERSITY OF SCIENCE AND TECHNOLOGY, Hong Kong (CN)

(72) Inventors: Karl Wah Keung TSIM, Hong Kong (CN); Tingxia DONG, Hong Kong (CN); Ka Wing LEUNG, Hong Kong (CN); Shengying LIN, Hong Kong (CN); Suisui GUO, Hong Kong (CN)

(21) Appl. No.: 19/141,902

(22) PCT Filed: Dec. 25, 2023
(86) PCT No.: PCT/CN2023/141534

§ 371 Date: Jun. 20, 2025

(30) Foreign Application Priority Data
Dec. 30, 2022 (CN) 202211731130.3

(51) Int. Cl. C07D 403/04; A61K 31/403

(52) U.S. Cl. CPC C07D 403/04; A61K 31/403

(57) ABSTRACT

Provided are a stilbene compound and a preparation method therefor and a use thereof. The structure of the stilbene compound is as shown in general formula (I), wherein X is halogen. Compared with resveratrol, the stilbene compound can more effectively inhibit binding of VEGF and a receptor VEGFR-1 so as to inhibit generation of new blood vessels.

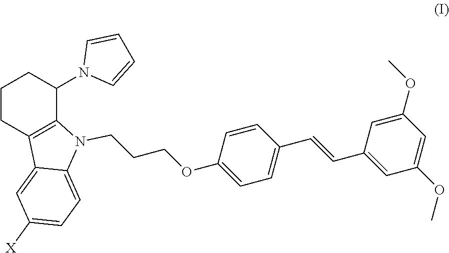

### RELATED APPLICATIONS

This application is an U.S. national phase application under 35 U.S.C. § 371 based upon international patent application No. PCT/CN2023/141534, filed on Dec. 25, 2023, which itself claims priority to Chinese patent application No. 202211731130.3, filed on Dec. 30, 2022, entitled “STILBENE COMPOUND, PREPARATION METHOD THEREOF, AND USE THEREOF”. The contents of the above identified applications are hereby incorporated herein in their entireties by reference.

### TECHNICAL FIELD

The present application is related to the fields of biology, medicine, and organic synthesis technology, and specifically is related to stilbene compounds, preparation methods thereof, and use thereof, particularly to stilbene compounds, preparation methods thereof, and use thereof in preparation of drugs for treating diseases caused by angiogenesis or blood vessel growth, as well as anti-tumor drugs.

### BACKGROUND

Angiogenesis refers to the formation of new blood vessels from pre-existing capillaries or post-capillary venules. Angiogenesis is a natural physiological phenomenon in the human body and plays a critical role in embryogenesis and tissue repair following injury. Under normal physiological conditions, a relative balance exists between molecules that inhibit and promote angiogenesis. However, under pathological conditions, angiogenesis becomes uncontrolled, leading to abnormal growth. Pathological angiogenesis has been confirmed to be closely associated with the occurrence and progression of numerous diseases, including tumors, ocular neovascularization, arthritis, skin disorders, and atherosclerosis. In addition, the proliferation and metastasis of tumors are believed to be dependent on angiogenesis. Therefore, research on anti-angiogenic drugs holds great importance for exploring treatment methods for these diseases.

Anti-angiogenesis is an extremely complex process, involving the modulation of angiogenesis regulatory factors, inhibition of vascular basement membrane and extracellular matrix degradation, and inhibition of endothelial cell division, migration, and proliferation. Vascular endothelial growth factor (VEGF) is regarded as the most critical pro-angiogenic factor. Anti-angiogenic and anti-tumor drugs targeting VEGF and its receptor VEGFR have received significant research interest in recent years.

It has been reported that the natural stilbene compound resveratrol exhibits a wide range of biological activities, including the inhibition of vascular endothelial cell proliferation, migration, tubule formation, and wound healing. To further explore the anti-angiogenic activity of stilbene compounds, the technical solutions of the present application is proposed.

### SUMMARY

In view of the above, a stilbene compound, a preparation method thereof, and use thereof are provided according to various embodiments of the present application. The technical solutions are as follow:

An embodiment of the present application provides a stilbene compound having the structure represented by Formula I:

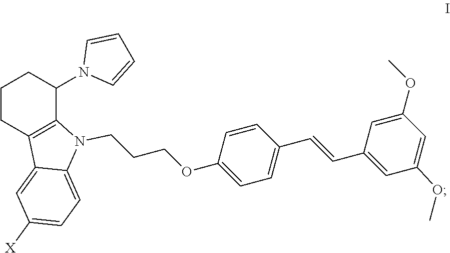

wherein x is a halogen.

An embodiment of the present application provides a method for preparing a stilbene compound, including the following steps:

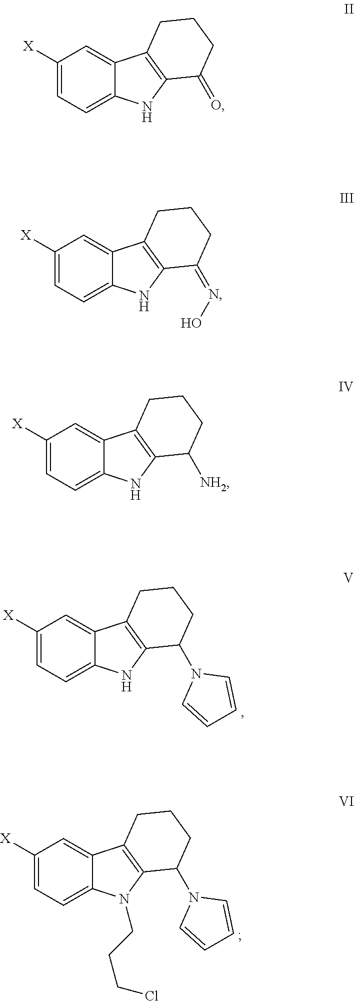

reacting compound II with hydroxylamine to obtain compound III;
subjecting compound III to a reduction reaction to obtain compound IV;
subjecting compound IV to a cyclization reaction to obtain compound V;
subjecting compound V to a substitution reaction to obtain compound VI;
reacting compound VI with pterostilbene to obtain the stilbene compound.

In some embodiments of the present application, the preparation method has one or more following technical features:
(1) the conditions for subjecting compound V to the substitution reaction to obtain compound VI include: reacting in the presence of sodium hydride at a temperature in a range from 0° C. to 20° C.;
(2) the conditions for reacting compound VI with pterostilbene include: reacting in the presence of sodium hydride at a temperature in a range from 20° C. to 30° C.

An embodiment of the present application provides the use of the stilbene compound in preparation of a drug for treating a disease caused by angiogenesis or blood vessel growth.

In some embodiments of the present application, in the use, the disease caused by angiogenesis or blood vessel growth includes one or more following diseases:
(1) a tumor; further, the tumor is a solid tumor;
(2) leukemia;
(3) psoriasis;
(4) Paget's disease;
(5) benign vascular proliferation;
(6) arthritis;
(7) atherosclerosis;
(8) a neovascular eye disease; further, the neovascular eye disease is a primary or secondary neovascular eye disease.

An embodiment of the present application provides the use of the stilbene compound in preparation of an anti-tumor drug.

In some embodiments of the present application, in the use, the tumor is a solid tumor, and is further a primary or secondary solid tumor.

In some embodiments of the present application, in the use, the tumor is lymphoma or myeloma.

An embodiment of the present application provides a pharmaceutical composition including an active pharmaceutical ingredient and a pharmaceutically acceptable component.

The active pharmaceutical ingredient is the stilbene compound provided in the first aspect of the present application.

In some embodiments of the present application, the pharmaceutical composition is in a dosage form selected from: capsules, tablets, microencapsulated tablets, injections, suppositories, sprays, or patches.

The details of one or more embodiments of the present application are set forth in the following description. Other features, objectives, and advantages of the present application will become apparent from the specification and claims.

### BRIEF DESCRIPTION OF THE DRAWINGS

In order to describe the technical solutions of the embodiments of the present application or the related art more clearly, the drawings used in the embodiments or the related art will be described briefly. Apparently, the following described drawings are merely for some embodiments of the present application, and other drawings can be derived according to these drawings by those of ordinary skill in the art without any creative effort.

FIG. 1 shows a synthetic route for a stilbene compound in an embodiment of the present application, wherein the reaction conditions for each step are as follows: (a) in the presence of hydroxylamine, pyridine, and ethanol, react under reflux for 1 hour; (b) in the presence of lithium aluminum hydride, diethyl ether, dichloromethane, and tetrahydrofuran, react at 0° C. to 50° C. for 10 hours; (c) in the presence of 2,5-dimethoxytetrahydrofuran and acetic acid, react at 60° C. for 6 hours; (d) in the presence of sodium hydride and tetrahydrofuran, react at 0° C. to room temperature for 16 hours; (e) in the presence of sodium hydride and tetrahydrofuran, react at room temperature for 16 hours.

FIG. 2 shows the ¹H NMR spectrum of compound RE-1 prepared in Example 1 of the present application.

FIG. 3 shows the high-performance liquid chromatograph (HPLC) of compound RE-1 prepared in Example 1 of the present application.

FIG. 4A and FIG. 4B show a photographic comparison and a quantitative comparison chart of the inhibition of HaCaT cell migration in the scratch assay by compound RE-1 prepared in Example 1 of the present application and resveratrol.

FIG. 5A and FIG. 5B show quantitative comparison charts of the potential cytotoxicity of compound RE-1 prepared in Example 1 of the present application and the positive control resveratrol in the MTT assay.

FIG. 6A and FIG. 6B show a photographic comparison and a quantitative comparison chart of the inhibition of reactive oxygen species (ROS) by compound RE-1 prepared in Example 1 of the present application and the positive control resveratrol.

FIG. 7A and FIG. 7B show comparative graphs created by molecular docking software, showing compound RE-1 prepared in Example 1 of the present application and the positive control resveratrol targeting the VEGFR-1 receptor binding domain of VEGF protein.

### DETAILED DESCRIPTION

The present application will be further described in details with reference to the implementations, embodiments, and drawings. It should be understood that the embodiments are for illustration only and not intended to limit the scope of the present application. In addition, it should be understood that after reading the content of the present application, those skilled in the art can make various changes or modifications to the present application, and equivalent forms obtained also fall within the protection scope of the present application.

Unless defined otherwise, all scientific and technical terms used herein have the same meaning as commonly understood by those skilled in the art of the present application. The terms used in the present application are for the purpose of describing specific embodiments only and are not intended to limit the protection scope of the present application.

### Terminology

Unless otherwise specified or there is no contradiction, the terms or phrases used herein have the following meanings:

As used herein, the terms “and/or”, “or/and”, “as well as/or”, when used in a list of two or more associated items, mean that any one of the items can be selected, and any or all combinations of the items can be selected. The any or all combinations include the combination of any two of the associated listed items, the combination of even more associated listed items, or the combination of all associated listed items. It should be noted that in the present application, when at least three items are connected by at least two conjunction combinations selected from “and/or”, “or/and”, “as well as/or”, the technical solutions undoubtedly include the technical solutions that are all connected by “logical AND” and also undoubtedly includes the technical solutions that are all connected by “logical OR”. As example, “A and/or B” includes three parallel solutions, i.e., A, B, and A+B. As another example, “A, and/or B, and/or C, and/or D” includes any one of A, B, C, or D (i.e., the technical solutions connected by “logical OR”), and combinations of any or all of A, B, C, D, such as a combination of any two or three of A, B, C, D and a combination of A, B, C, and D (i.e., the technical solutions connected by “logical AND”).

Herein, “preferably”, “better”, “preferred”, etc., are merely for describing implementations or embodiments having good effects, and do not constitute a limitation to the protection scope of the present application.

In the present application, “further”, “furthermore”, “particularly”, etc., are used for description purposes, indicating differences in content, but should not be construed as limiting the protection scope of the present application.

In the present application, an open-ended description for the technical features includes not only a close-ended technical solution consisting of the recited technical features, but also an open-ended technical solution including the recited technical features.

In the present application, unless otherwise specified, when a numerical interval (i.e., a numerical range) is referred to, the suitable values are considered as continuously distributed within this numerical interval and include both endpoints of the numerical range (i.e., the minimum value and the maximum value), as well as every value between the two endpoints. Unless otherwise specified, when a numerical interval is referred to as encompassing only integers within the numerical interval, it includes the integers at both endpoints of the numerical interval, and every integer between the endpoints. Furthermore, when multiple ranges are provided to describe a feature or a characteristic, these ranges can be combined. In other words, ranges disclosed herein are to be construed to include any or all sub-ranges subsumed therein, unless otherwise specified.

Unless otherwise defined, in the present application, a temperature parameter is allowed to involve a constant-temperature treatment, or with variation in a certain temperature interval. The constant-temperature treatment allows the temperature to fluctuate within the precision range under the control of an instrument, such as fluctuate within the ranges of ±5° C., ±4° C., ±3° C., ±2° C., ±1° C.

In the present application, the masses or weights are in units well known in the chemical industry, such as μg, mg, g, kg, etc.

Stilbene compounds are widely present in nature and exhibit a broad spectrum of biological activities, including anti-inflammatory, anti-oxidation, anti-bacteria, anti-allergy, anti-thrombosis, anti-sugar, anti-tumor, etc. However, there are few reports on the VEGF inhibitory effects of stilbene compounds.

The present application provides a stilbene compound with potent VEGF inhibitory effect, surpassing that of the natural stilbene compound resveratrol.

In some embodiments, the structure of the stilbene compound is represented by Formula I.

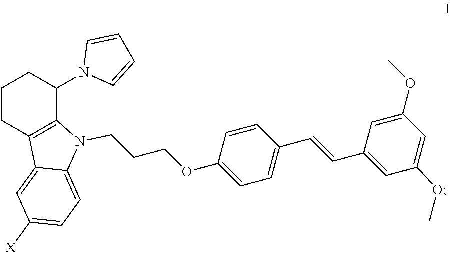

wherein x is a halogen.

In some embodiments, x is Br.

In some embodiments, the stilbene compound can inhibit ROS activity induced by VEGF.

In some embodiments, the stilbene compound can effectively inhibit the proliferation of vascular endothelial cells and wound healing.

In some embodiments, the stilbene compound can effectively inhibit the formation of new blood vessels.

An embodiment of the present application provides a method for preparing a stilbene compound. The preparation method of the present application involves a rationally designed synthetic route, readily available starting materials, inexpensive reagents, and simple post-processing, which can effectively reduce costs.

In some embodiments, the method for preparing the stilbene compound includes the following steps:

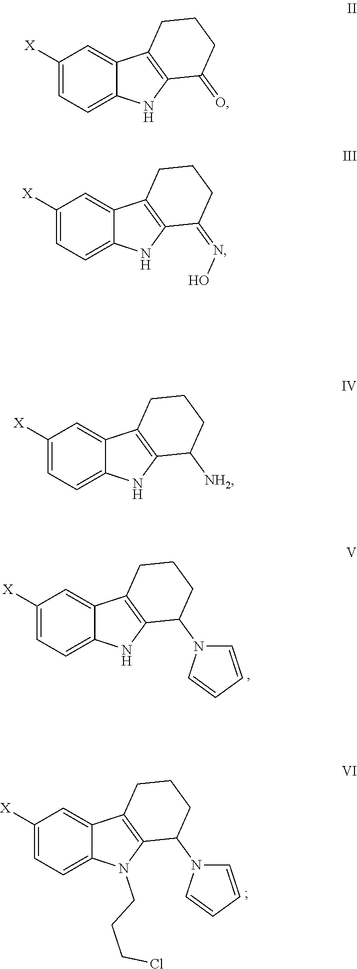

reacting compound II with hydroxylamine to obtain compound III;
subjecting compound III to a reduction reaction to obtain compound IV;
subjecting compound IV to a cyclization reaction to obtain compound V;
subjecting compound V to a substitution reaction to obtain compound VI;
reacting compound VI with pterostilbene to obtain the stilbene compound.

In some embodiments, the reaction conditions for reacting compound II with hydroxylamine to obtain compound III include: reacting in the presence of ethanol and pyridine; further, the reaction temperature is in a range from 65° C. to 75° C., and the reaction time is in a range from 1 hour to 3 hours.

In some embodiments, the conditions for the reduction reaction include: reacting with lithium aluminum hydride as a reducing agent in diethyl ether, dichloromethane, and tetrahydrofuran as solvents at a temperature in a range from 45° C. to 55° C. In some embodiments, the conditions for the reduction reaction include: adding diethyl ether, dichloromethane, tetrahydrofuran, and lithium aluminum hydride at 0° C., followed by heating to a temperature in a range from 45° C. to 55° C. for the reaction. In some embodiments, the reaction time of the reduction reaction is 10 hours to 15 hours.

In some embodiments, the conditions for the cyclization reaction include: reacting with 2,5-dimethoxytetrahydrofuran in the presence of acetic acid; further, the reaction temperature is in a range from 60° C. to 70° C.; and furthermore, the reaction time is 6 hours to 8 hours.

In some embodiments, the conditions for the substitution reaction include: reacting in the presence of sodium hydride at a temperature in a range from 0° C. to 20° C. Further, the reaction time is 16 hours to 20 hours.

In some embodiments, the conditions for reacting compound VI with pterostilbene include: adding sodium hydride at 0° C., followed by heating to a temperature in a range from 20° C. to 30° C. for the reaction. Further, the reaction time is 16 hours to 20 hours.

An embodiment of the present application provides the use of the stilbene compound described in any one of the above technical solutions in preparation of a drug for treating a disease caused by angiogenesis or blood vessel growth.

In some embodiments of the present application, the disease caused by angiogenesis or blood vessel growth includes one or more following diseases:
(1) a tumor; further, the tumor is a solid tumor;
(2) leukemia;
(3) psoriasis;
(4) Paget's disease;
(5) benign vascular proliferation;
(6) arthritis;
(7) atherosclerosis;
(8) a neovascular eye disease; further, the neovascular eye disease is a primary or secondary neovascular eye disease.

An embodiment of the present application provides the use of the stilbene compound described in any one of the above technical solutions in preparation of an anti-tumor drug.

In some embodiments of the present application, the tumor is a solid tumor, and is further a primary or secondary solid tumor.

In some embodiments of the present application, the tumor is a lymphoma or myeloma.

An embodiment of the present application provides a pharmaceutical composition including an active pharmaceutical ingredient and a pharmaceutically acceptable component;

The active pharmaceutical ingredient is the stilbene compound provided in the first aspect of the present application.

In some embodiments of the present application, the pharmaceutical composition is in a dosage form selected from: capsules, tablets, microencapsulated tablets, injections, suppositories, sprays, or patches.

The following are some specific examples.

The experimental parameters not specified in the following specific examples should refer first to the guidance given in the present application, or to the experimental manuals in the art, or to the other experimental methods known in the art, or to the experimental conditions recommended by manufacturers. The raw materials and reagents involved in the following specific examples are commercially available or can be prepared by those skilled in the art using known methods.

### Example 1: Preparation of Stilbene Compound RE-1

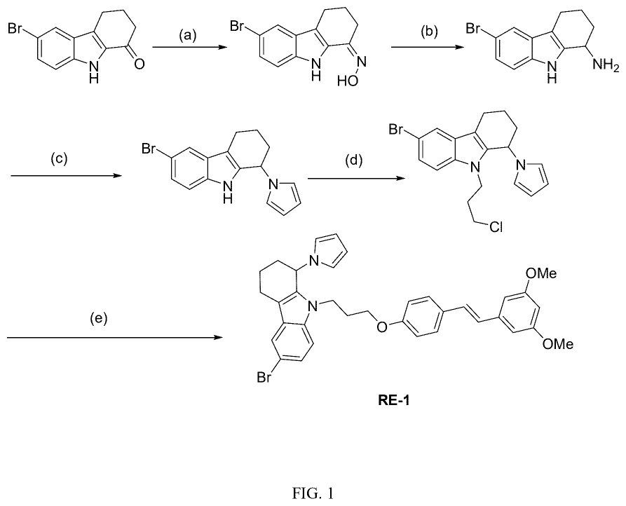

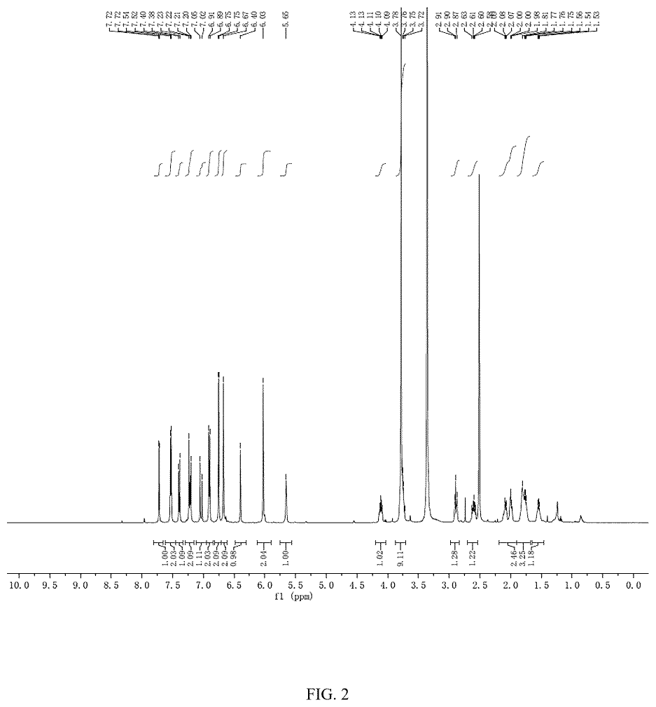

The synthesis was carried out according to the synthetic route shown in FIG. 1, and the resulting compound RE-1 was subjected to NMR analysis. The obtained ¹H NMR spectrum is shown in FIG. 2.

¹H NMR: (400 MHz, DMSO-d₆) δ 1.77-1.81 (1H, m), 1.98-2.00 (3H, m), 2.07-2.09 (2H, m), 2.58-2.63 (1H, m), 2.87-2.91 (1H, m), 3.72-3.78 (9H, m), 4.09-4.13 (1H, m), 5.65 (1H, s), 6.03 (2H, s), 6.40 (1H, s), 6.67-6.75 (4H, m), 6.89 (2H, d, J=6.5 Hz), 7.02 (1H, d, J=10.2 Hz), 7.20-7.22 (2H, m), 7.23 (1H, d, J=10.2 Hz), 7.38 (2H, d, J=6.5 Hz), 7.72 (1H, d, J=2.2 Hz).

The resulting compound was subjected to HPLC detection, indicating a purity of above 97%. The detection result is shown in FIG. 3. The detection method was as follows: The sample was dissolved in 4 mL of 50% methanol, sonicated for 15 minutes, and then centrifuged at 1000×g for 5 minutes to obtain the supernatant. Prior to sample injection, the supernatant was filtered through a 0.45 μm microporous membrane, and the filtrate was used for sample injection and analysis. The analytical instrument used was an Agilent HPLC system equipped with an autosampler and a binary pump. The chromatographic column was an Agilent Grace VisionHT C18 column (4.6×250 mm, 5 μm). The mobile phase consisted of acetonitrile (solvent A) and 0.2% formic acid water solution (solvent B), with a flow rate of 1 mL/min and a column temperature of room temperature. Gradient elution was performed with the mobile phase composition as follows: 0-60 minutes, 10-35% solvent A; 60-96 minutes, 35-100% solvent A. The injection volume was 10 μL, and the detection wavelength was 280 nm.

### Example 2: Cell Scratch Assay

### 2.1. Method

HaCaT cells were inoculated at a density of 20×10⁴ cells/well in a 12-well plate and cultured overnight to allow cell adhesion to the wall. When the cells reached 80% confluence, a 200 μL pipette tip was used to transversely scratch a wound through the center of each well. The culture medium was discarded. The wells were washed once with PBS, supplemented with culture medium including 10 ng/mL human vascular endothelial growth factor (VEGF). 200 μg/mL Avastin, different concentrations of resveratrol (0.3 μM, 1 μM, 3 μM), and different concentrations of compound RE-1 (0.3 μM, 1 μM, 3 μM) were respectively administrated to the wells. Additionally, a blank group (with no drug but an equal volume of solvent) and a control group (with no drug) were established. A microscope equipped with a camera was used to capture 50× magnification images at 0 hours and 24 hours respectively, monitoring the cell layer coverage variation in each well. The wound recovery rate was quantified using the TScratch software, calculated according to the formula: Recovery rate (%)=(At₀−At₂₄)/At₀×100%, where At₀ is the scratch area measured at hour 0 after drug treatment, and At₂₄ is the scratch area measured at hour 24 after drug treatment.

### 2.2. Result and Conclusion

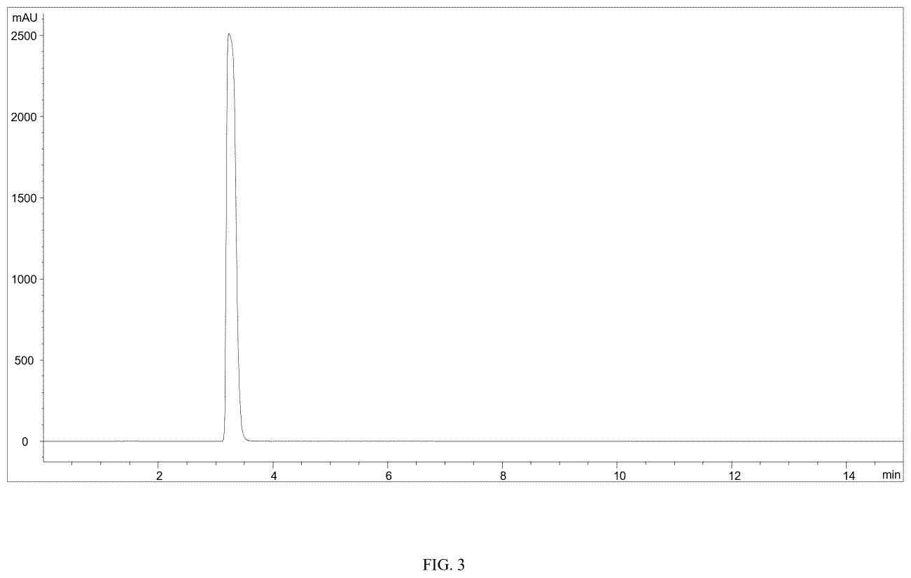

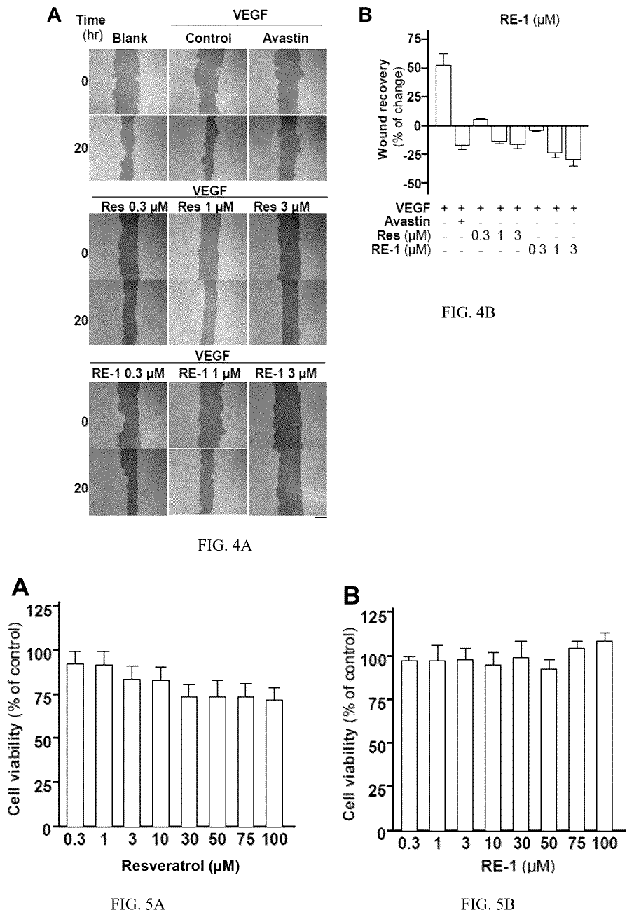

FIG. 4A shows photographs of the inhibition of HaCaT cell migration in the scratch assay by resveratrol and compound RE-1. The labels in the figures have the following meanings: Time: time after drug treatment; Blank: blank group; Control: control group; Avastin: positive control group treated with Avastin; Res 0.3 μM, Res 1 μM, Res 3 μM: control groups treated with different concentrations of resveratrol; RE-1 0.3 μM, RE-1 1 μM, RE-1 3 μM: experimental groups treated with different concentrations of compound RE-1.

FIG. 4B shows a quantitative chart showing the inhibition of HaCaT cell migration in the scratch assay by resveratrol and compound RE-1, where “Wound recovery (% of change)” represents the degree of wound closure (%).

As shown in FIGS. 4A and 4B, compound RE-1 effectively inhibits the proliferation of HaCaT cells and wound healing, with effects superior to those of resveratrol.

### Example 3: Cytotoxicity Assay

### 3.1. Method

The tetrazolium salt (MTT) colorimetric assay was used to evaluate the cytotoxic effects of compound RE-1 and resveratrol on human HaCaT cells. HaCaT cells were inoculated at a density of 5.0×10³ cells/mL in a 96-well culture plate, with 100 μL per well. When the cells reached 80% confluence, different concentrations of resveratrol and compound RE-1 (0.3, 1, 3, 10, 30, 50, 75, 100 μM) were respectively added to the wells. After 48 hours of treatment, 10 μL of MTT solution was added to each well, and the cells were incubated at 37° C. for an additional 4 hours. The culture was then terminated, the supernatant was discarded, and 150 μL of DMSO was added to each well. The plate was shaken for 10 minutes for full dissolution. The absorbance (OD) of each well was measured at 490 nm using an ELISA reader. The wells without drug treatment served as controls, and cell viability was calculated using the formula: Cell viability

$$( \% ) = ( {OD}_{sample} - {OD}_{control} ) / {OD}_{control} \times 100 \% .$$

### 3.2. Result and Conclusion

FIG. 5A shows the cell viability of HaCaT cells treated with resveratrol, where “Cell viability (% of change)” represents the cell viability (%), and “Resveratrol (μM)” represents the amount of resveratrol administered.

FIG. 5B shows the cell viability of HaCaT cells treated with compound RE-1, where “Cell viability (% of change)” represents the cell viability (%), and “RE-1 (μM)” represents the amount of compound RE-1 administered.

As shown in FIGS. 5A and 5B, neither compound RE-1 nor resveratrol exhibited significant cytotoxicity. It can be noted that the cell viability at high concentrations of compound RE-1 was higher than that at equivalent concentrations of resveratrol, indicating that compound RE-1 is safer than resveratrol.

### Example 4: ROS Inhibition Effect

### 4.1. Experiment Principle

The binding of VEGF to its receptor VEGFR-1 promotes ROS activity, which in turn stimulates signaling pathways and induces the formation of new blood vessels. Therefore, inhibiting VEGF-induced ROS can effectively suppress the formation of new blood vessels.

### 4.2. Experiment Procedure

ROS formation was assessed using the DCF-DA assay, using the kit purchased from Sigma Company. According to the supplier's protocol, HaCaT cells were cultured with VEGF for 48 hours, and treated with the positive control Avastin, different concentrations of resveratrol (0.3 μM, 1 μM, 3 μM), or different concentrations of compound RE-1 (0.3 μM, 1 μM, 3 μM). Additionally, a blank group (treated with no drug but an equal volume of solvent) and a control group (treated with no drug) were established. Subsequently, DCF-DA reagent (20 μM) was added, the cells were incubated for 30 minutes, and excess DCF-DA was removed. The cells were then analyzed under a confocal microscope, and the inhibition rate was quantified using the ImageJ software.

### 4.3. Experiment Result

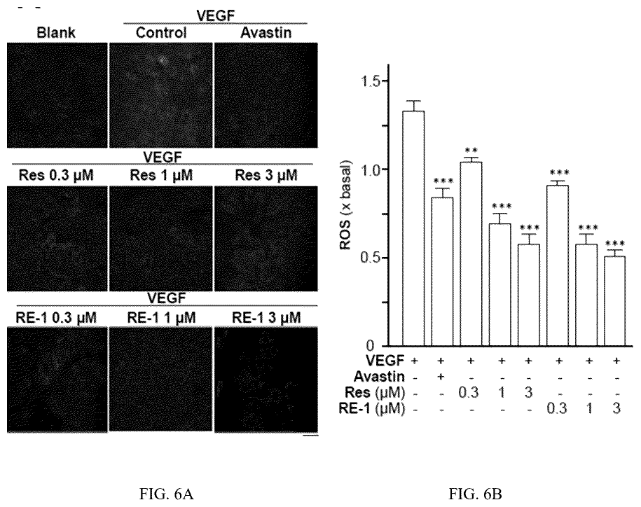

FIG. 6A shows photographs of ROS inhibition by compound RE-1 and resveratrol. The labels in the figures have the following meanings: Time: time after treatment; Blank: blank group; Control: control group; Avastin: positive control group treated with Avastin; Res 0.3 μM, Res 1 μM, Res 3 μM: control groups treated with different concentrations of resveratrol; RE-1 0.3 μM, RE-1 1 μM, RE-1 3 μM: experimental groups treated with different concentrations of compound RE-1.

FIG. 6B shows a quantitative comparison chart of ROS inhibition by compound RE-1 and resveratrol, where “ROS (×basal)” represents the relative ROS expression level.

As shown in FIGS. 6A and 6B, compound RE-1 exhibits a favorable dose-response relationship at relatively low concentrations, such as 0.3 μM to 3 μM, and demonstrates superior ROS inhibition compared to resveratrol within this concentration range.

### Example 5: Molecular Docking Experiment

### 5.1. Experiment Principle

Vascular endothelial growth factor (VEGF) is a critical protein for vascular endothelial cells. Under normal conditions, VEGF binds to the VEGFR-1 receptor, providing a material basis for subsequent signaling pathways. In the VEGF protein, the binding domain that binds to the VEGFR-1 receptor is amino acid residues 1-165. Theoretically, a chemical compound that can target this binding domain and competitively bind to VEGF can effectively inhibit the binding between VEGF and its receptor VEGFR, thereby affecting downstream pathways and ultimately suppressing angiogenesis. This binding domain was designated as the target for the molecular docking.

### 5.2. Experiment Procedure and Result

Using the SeeSAR software, the VEGFR-1 receptor binding domain (amino acid residues 1-165) of the VEGF protein (PDB code: 1FLT) was selected as the target to evaluate the binding affinity of compound RE-1 and resveratrol to this target. The docking software SeeSAR was used to calculate the target binding energy, where a lower binding energy indicates a more stable binding between the chemical molecule and the target, suggesting a stronger potential inhibitory effect.

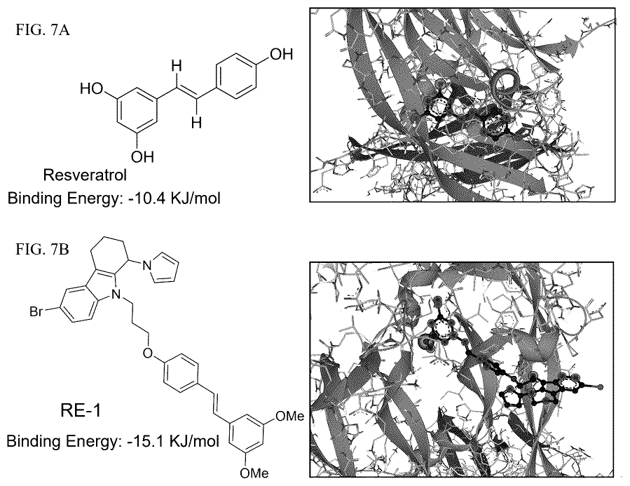

The results are shown in FIGS. 7A and 7B, demonstrating that resveratrol effectively binds to the VEGF target (FIG. 7A), with a predicted binding energy of −10.4 kJ/mol. According to analysis through the molecular docking software, the binding energy of compound RE-1 to the target is about −15.1 kJ/mol (FIG. 7B), indicating that compound RE-1 can more efficiently target the VEGF protein, thereby preventing its binding with the VEGFR-1 receptor, leading to more effective inhibition of VEGF and more effective inhibition of angiogenesis. These findings are consistent with the experiment results from Examples 2, 3, and 4.

The technical features of the above implementations and embodiments can be combined arbitrarily. In order to make the description concise, not all possible combinations of the technical features are described in the implementations and embodiments. However, as long as there is no contradiction in the combination of these technical features, the combinations should be considered as in the scope of the present application.

The above-described embodiments are only several implementations of the present application, which facilitate specific and detailed understanding of the technical solutions of the present application, but they should not be understood as limiting the patent protection scope of the present application. It should be noted that various modifications and improvements can be made by those skilled in the art without departing from the concept of the present application, which are all within the protection scope of the present application. In addition, it should be understood that after reading the above teachings of the present application, those skilled in the art can make various changes or modifications to the present application, and equivalent forms obtained also fall within the protection scope of the present application. It should also be understood that the technical solutions obtained by those skilled in the art through logical analysis, reasoning or limited experiments on the basis of the technical solutions provided by the present application are all within the protection scope of the appended claims of the present application. Accordingly, the patent protection scope of the present application shall be defined by the appended claims, and the description and the accompany drawings may be used to interpret the content of the claims.

1. A stilbene compound having a structure represented by Formula I: 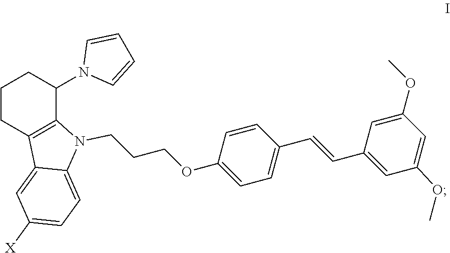
wherein x is a halogen.

2. A method for preparing the stilbene compound according to claim 1, comprising following steps: 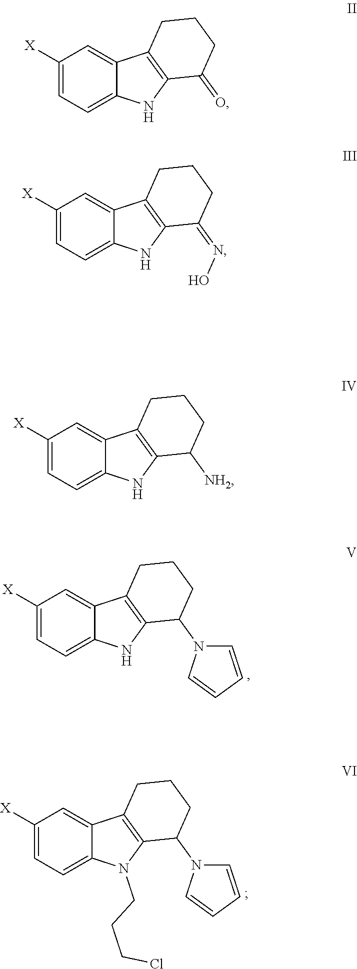
reacting compound II with hydroxylamine to obtain compound III;
subjecting compound III to a reduction reaction to obtain compound IV;
subjecting compound IV to a cyclization reaction to obtain compound V;
subjecting compound V to a substitution reaction to obtain compound VI;
reacting compound VI with pterostilbene to obtain the stilbene compound.

3. The method according to claim 2, satisfying one or more following technical features:
(1) the substitution reaction comprises: reacting in the presence of sodium hydride at a temperature in a range from 0° C. to 20° C.;
(2) reacting compound VI with pterostilbene comprises: reacting in the presence of sodium hydride at a temperature in a range from 20° C. to 30° C.

4-8. (canceled)

9. A pharmaceutical composition comprising an active pharmaceutical ingredient and a pharmaceutically acceptable component, wherein the active pharmaceutical ingredient is the stilbene compound according to claim 1.

10. The pharmaceutical composition according to claim 9, wherein the pharmaceutical composition is in a dosage form selected from capsules, tablets, microencapsulated tablets, injections, suppositories, sprays, or patches.

11. A method for treating a disease caused by angiogenesis or blood vessel growth, the method comprising administering to a patient in need thereof a therapeutically effective amount of the stilbene compound according to claim 1.

12. The method according to claim 11, wherein the disease caused by angiogenesis or blood vessel growth comprises one or more following diseases:
(1) a tumor;
(2) leukemia;
(3) psoriasis;
(4) Paget's disease;
(5) benign vascular proliferation;
(6) arthritis;
(7) atherosclerosis;
(8) a neovascular eye disease.

13. The method according to claim 12, wherein the tumor is a solid tumor.

14. The method according to claim 13, wherein the solid tumor is a primary solid tumor or a secondary solid tumor.

15. The method according to claim 12, wherein the tumor is lymphoma or myeloma.

16. The method according to claim 12, wherein the neovascular eye disease is a primary or secondary neovascular eye disease.
